(19) United States
(12) Patent Application Publication
(10) Pub. No.: US 2025/0025244 A1
(43) Pub. Date: Jan. 23, 2025

(54) SURGICAL END EFFECTOR WITH MONOPOLAR HOOK AND RELATED METHODS

(71) Applicant: Cilag GmbH International, Zug (CH)

(72) Inventors: Niko J. Murrell, Evandale, OH (US); Joseph M. Nath, West Chester, OH (US); Kevin M. Montgomery, Morrow, OH (US)

(21) Appl. No.: 18/663,874

(22) Filed: May 14, 2024

Related U.S. Application Data
(60) Provisional application No. 63/528,136, filed on Jul. 21, 2023.

(51) Int. Cl. A61B 34/30; A61B 18/00; A61B 18/12; A61B 18/14

(52) U.S. Cl. CPC A61B 34/30; A61B 18/12; A61B 2018/00184; A61B 2018/00595; A61B 2018/1422; A61B 2034/302; A61B 2034/306

(57) ABSTRACT

An apparatus includes a conductive cable, a pulley assembly, and an end effector electrically coupled to the conductive cable. The pulley assembly includes an inner body defining a cable pathway, an outer body fixed to the inner body, and a distal cap defining an interior. The end effector includes a distal hook that can deliver therapeutic energy to tissue, and a proximal body fixed relative to the dismal hook. The proximal body defines a conductive wire channel in communication with the cable pathway defined by the inner body. The conductive cable extends though the cable pathway of the inner body and into the conductive wire channel. The proximal body of the end effector defines a first port located within the interior of the distal cap, where the first port extends from an outer surface of the proximal body into the conductive wire channel.

### PRIORITY

This application claims priority to U.S. Provisional Pat. App. No. 63/528,136, entitled “Robust Monopolar Hook for Robotic Surgical Applications”, filed on Jul. 21, 2023.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

In some instances, an instrument of a robotic surgical system may deliver therapeutic energy onto tissue. In such instances, it may be desirable to ensure such therapeutic energy is controllably delivered. Additionally, in some instances, an instrument of robotic surgical system may experience a large external force. In such instances it may be desirable to inhibit failure and/or plastic deformation of such an instrument.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first illustrative robotic system configured for a laparoscopic procedure;

FIG. 2 depicts a perspective view of a second illustrative robotic system;

FIG. 3 depicts an end elevational view of the robotic system of FIG. 2;

FIG. 4 depicts an end elevational view of the robotic system of FIG. 2 including an illustrative pair of robotic arms;

FIG. 5 depicts a partially exploded perspective view of the robotic arm of FIG. 4 having an instrument driver and a first illustrative surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7A depicts an enlarged perspective view of a second illustrative surgical instrument having an end effector in the form of a monopolar hook;

FIG. 7B depicts an enlarged perspective view of the surgical instrument of FIG. 7A, with select portions shown articulation about a distal pivot axis in phantom;

FIG. 7C depicts an enlarged perspective view of the surgical instrument of FIG. 7A, with select portions shown articulation about a proximal pivot axis in phantom;

FIG. 8 depicts an enlarged perspective view of the surgical instrument of FIG. 7A, with select options shown in phantom to better show various internal components;

FIG. 9 depicts a perspective view of an alternative end effector, distal pulley assembly, and conductive cable that may be readily incorporated into the surgical instrument of FIG. 7A;

FIG. 10 depicts a sectional view of the end effector, distal pulley assembly, and conductive cable of FIG. 9, taken along line 10-10 of FIG. 9;

FIG. 11 depicts a sectional view of the end effector, distal pulley assembly, and conductive cable of FIG. 9, taken along line 11-11 of FIG. 9;

FIG. 12 depicts a cross-sectional view of the end effector, distal pulley assembly, and conductive cable of FIG. 9, taken along line 12-12 of FIG. 9;

FIG. 13A depicts a perspective view of the end effector of FIG. 9;

FIG. 13B depicts a perspective view of the end effector of FIG. 9 with an inner body of the distal pulley assembly injection molded thereto;

FIG. 13C depicts a perspective view of end effector of FIG. 9 and the distal pulley assembly of FIG. 9;

FIG. 14A depicts a sectional view of the end effector of FIG. 9, taken along line 14A-14A of FIG. 13A;

FIG. 14B depicts a sectional view of view of the end effector of FIG. 9 with an inner body of the distal pulley assembly injection molded thereto, taken along line 14B-14B of FIG. 13B;

FIG. 14C depicts a sectional view of end effector of FIG. 9 and the distal pulley assembly of FIG. 9, taken along line 14C-14C of FIG. 13C;

FIG. 15A depicts a cross-sectional view of the end effector, distal pulley assembly, and conductive cable of FIG. 9, with a cap of the distal pulley assembly detached from the rest of the distal pulley assembly, with the conductive cable decoupled from both the end effector and the distal pulley assembly;

FIG. 15B depicts a cross-sectional view of the end effector, distal pulley assembly, and conductive cable of FIG. 9, with the cap of FIG. 15A detached from the rest of the distal pulley assembly, with the conductive cable coupled to both the end effector and the distal pulley assembly;

FIG. 15C depicts a cross-sectional view of the end effector, distal pulley assembly, and conductive cable of FIG. 9, with the cap of FIG. 15A detached from the rest of the distal pulley assembly, with the conductive cable coupled to both the end effector and the distal pulley assembly, with a dielectric cable sealant injected around an exterior of the conductive cable housed within the end effector and distal pulley assembly; and

FIG. 15D depicts a cross-sectional view of the end effector, distal pulley assembly, and conductive cable of FIG. 9, with the cap of FIG. 15A attached to the rest of the distal pulley assembly, with the conductive cable coupled to both the end effector and the distal pulley assembly.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

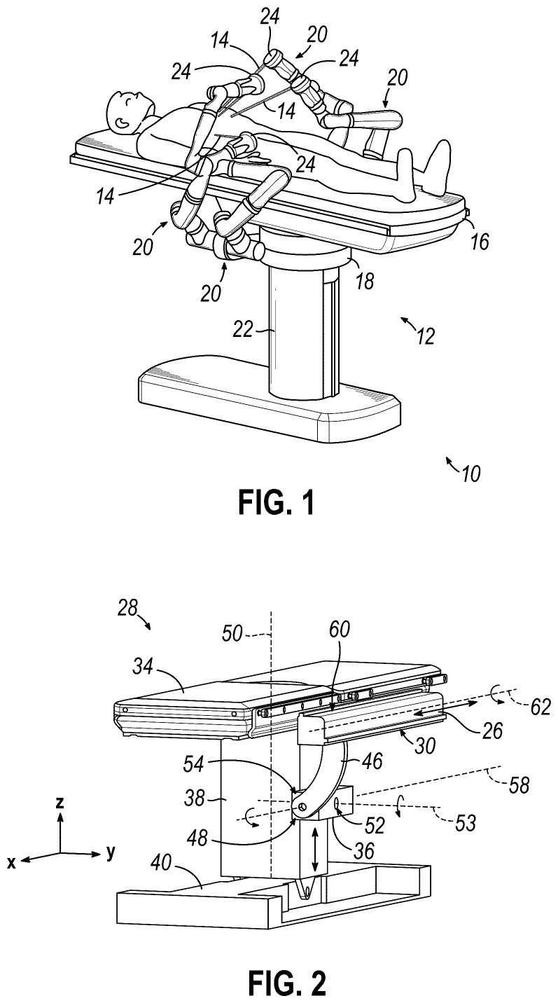

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

B. Example of Robotic System with Bar Carriage

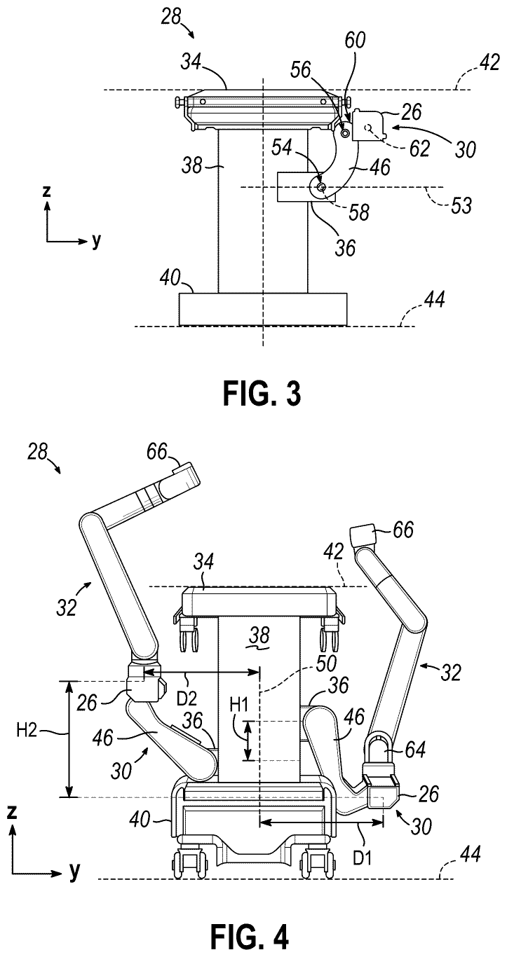

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

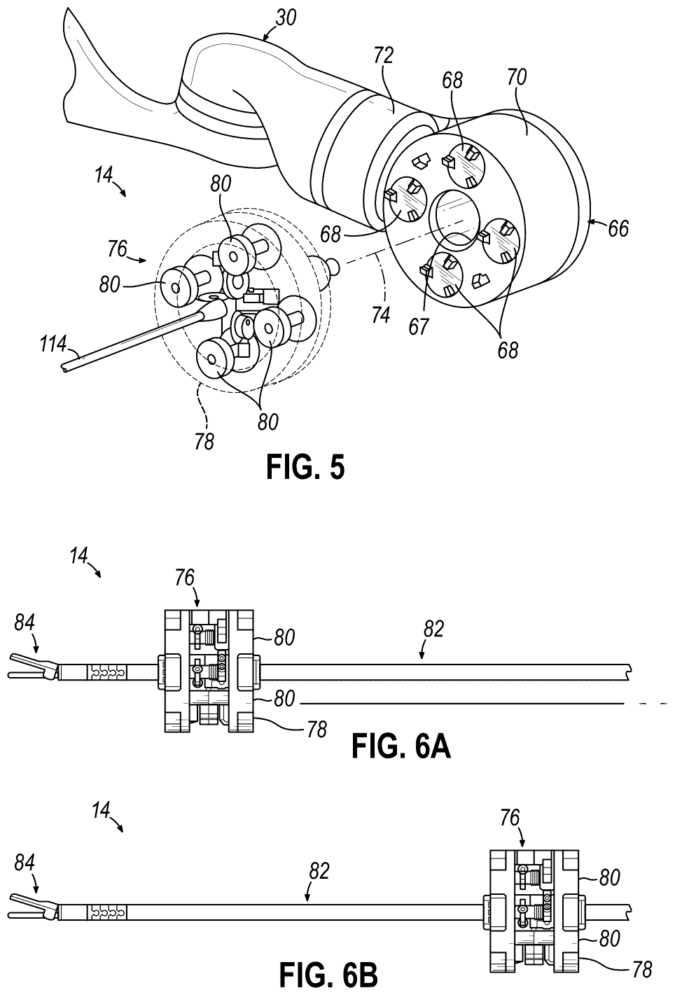

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

C. Example of Surgical Instrument with Instrument-Based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

II. Surgical Instrument with End Effector Having Monopolar Hook

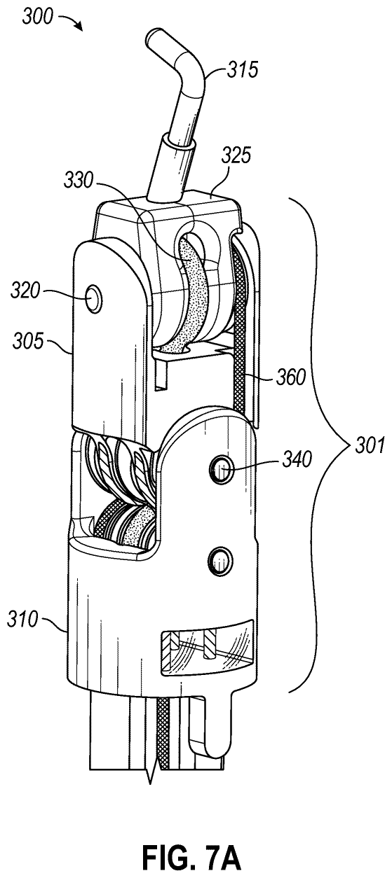

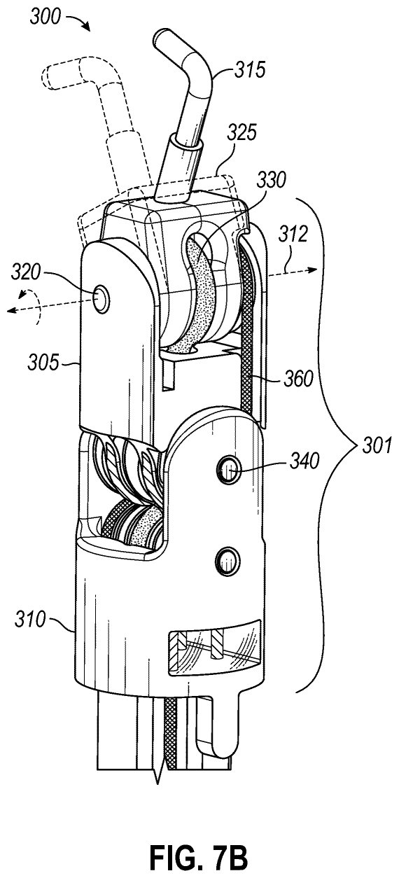

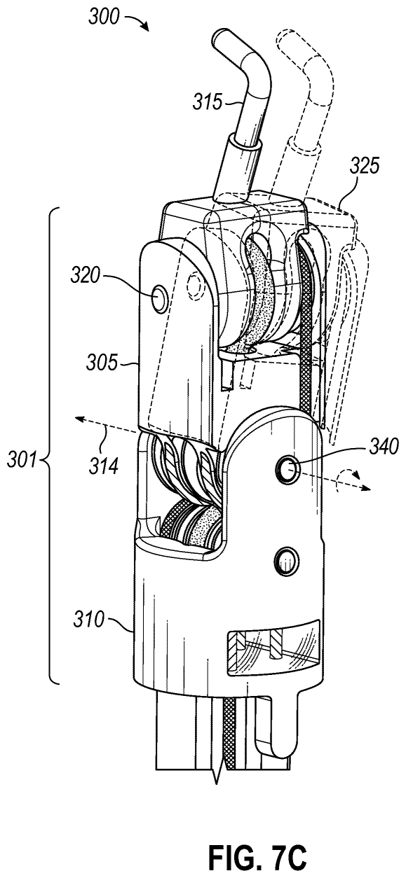

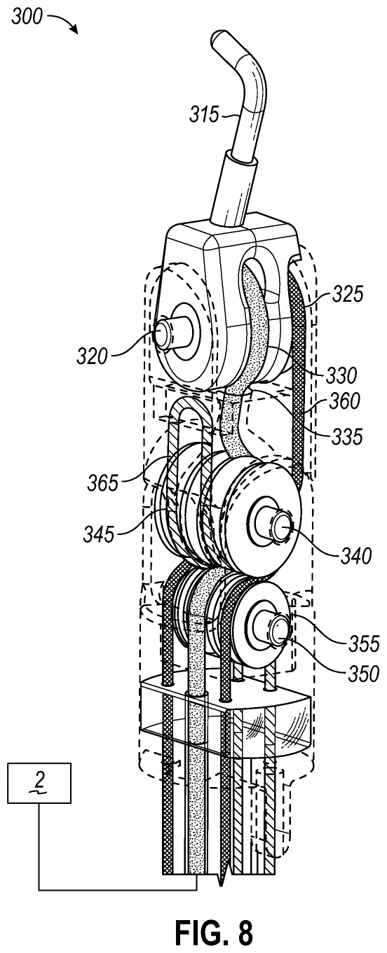

FIGS. 7A-8 show an illustrative monopolar medical instrument (300). Monopolar medical instrument (300) includes a wrist (301) and an end effector (315). Monopolar medical instruments (300) may be readily incorporated into robotic system (10, 28) described above. Therefore, while not explicitly shown, instrument (300) may include various suitable components configured to operatively couple with a suitable robotic arm (20, 32) such that instrument driver (24, 66) and/or other suitable components of robotic system (10, 28) may operatively control instrument (300) in accordance with the description herein. For example, instrument (300) may include an elongated shaft assembly (not shown) and instrument base (not shown), which may be substantially similar to elongated shaft assembly (82) and instrument base (76) described above.

Therefore, it should be understood that an elongated shaft assembly (not shown) may be attached to, and extend proximally from, wrist (301); while suitable components of monopolar medical instrument (300) may extend proximally along/within suitable portions of shaft assembly (not shown). Additionally, wrist (301) and end effector (315) may be advanced and retracted in accordance with the description herein, as well as rotated about a longitudinal axis of instrument (300) (e.g., the longitudinal axis of an elongated shaft assembly (82)). Of course, monopolar medical instrument (300) may include any other suitable robotic interface to thereby readily incorporate instrument (300) into other suitable robotic systems as would be apparent to one skilled in the art in view of the teachings herein.

In the current example, end effector (315) is a monopolar hook. As will be described in greater detail below, monopolar hook of end effector (315) is configured to deliver therapeutic monopolar energy to tissue. Such monopolar energy delivered by end effector (315) may be utilized to cauterize wounds, transect tissue, or any other suitable use as would be apparent to one skilled in the art in view of the teachings herein. As will also be described in greater detail below, when instrument (300) is operatively coupled to a suitable robotic system (e.g., robotics system (10, 28)), end effector (315) may be rotated about a distal pivot axis (312) and a proximal pivot axis (314). Monopolar hook of end effector (315) may be pivoted about axis (312, 314) in order to move, manipulate, and/or access tissue during illustrative use.

Wrist (301) includes a distal clevis (305) and a proximal clevis (310) which are rotatably coupled to each other via a proximal axel (340). As shown in FIG. 8, monopolar medical instrument (300) further includes the distal axle (320), a distal pulley (325), a conductive cable (330), a stationary redirect surface (335), proximal axle (340), a set of proximal pulleys (345), a proximal redirect axle (350), a set of proximal redirect pulleys (355), a first cable (360), and a second cable (365).

End effector (315) is fixedly attached to distal pulley (325). Further, distal pulley (325) is pivotally coupled to distal clevis (305) via distal axel (320). Therefore, both end effector (315) and distal pulley (325) are configured to pivot relative to distal clevis (305) about distal pivot axis (312) defined by distal axel (320) (see FIG. 7B).

First cable (360) traverses a first path within the wrist (301) to engage with proximal redirect pulleys (355), proximal pulleys (345), and distal pulley (325). In particular, first cable (360) is operatively coupled to distal pulley (325) such that movement of first cable (360) in a first direction pivots distal pulley (325) and end effector (315) in a first angular direction about distal axis (312) relative to distal clevis (305); while movement of first cable (360) in a second, opposite direction pivots distal pulley (325) and end effector (315) in a second, opposite, angular direction about distal axis (312) relative to distal clevis (305). Proximal redirect pulley(s) (355) and/or one or more proximal pulleys (345) suitably guide first cable (360) along the predetermined path to thereby suitable engage distal pulley (325) in accordance with the description herein. Of course, in some instances, use of proximal redirect pulley (355) and/or one or more proximal pulleys (345) in relation to its engagement with first cable (360) is optional.

In some instances, distal pulley (325) includes two separate pulleys that receive a respective cable (360) such that two cables (360) are utilized to pivot distal pulley (325) about distal axis (312). In such instances, a first cable (360) may be pulled in order to rotate distal pivot (325) in the first angular direction about axis (312), while a second cable (360) may be pulled in order to rotate distal pulley (325) in the second angular direction about axis (312). Of course, distal pulley (325) may include any other suitable features configured to suitably engage cable(s) (360) as would be apparent to one skilled in the art in view of the teachings herein.

One or more proximal ends (not shown) of first cable (360) may be operatively coupled to suitable components such that when instrument (300) is suitably coupled to robotic arm (20, 32), instrument driver (24, 66) (or other suitable components) is configured to actuate first cable (360) in accordance with the description herein. Therefore, when instrument (300) is operatively coupled to a robotic arm (20, 32) of robotic system (10, 28) robotic system (10, 28) may control the angular position of distal pulley (325) and end effector (315) about distal axis (312) relative to distal clevis (305).

Proximal pulleys (345) are fixedly attached to distal clevis (305). Further, proximal pulleys (345) and distal clevis (305) are pivotally coupled to proximal clevis (310) via proximal axel (340). Therefore, both proximal pulleys (345) and distal clevis (305) are configured to pivot about proximal pivot axis (314) defined by proximal axel (340) (see FIG. 7C). Since distal pulley (325) and end effector (315) are also pivotally attached to distal clevis (305) via distal axel (320), distal pulley (325) and end effector (315) also pivot with distal clevis (305) relative to proximal clevis (310) in accordance with the description herein.

Second cable (365) traverses a second path within wrist (301) so as to engage with proximal redirect pulleys (355) and proximal pulleys (435). In particular, second cable (365) is operatively coupled to proximal pulleys (345) such that movement of second cable (365) in a first direction pivots proximal pulley (345) and distal clevis (305) in a first angular direction about proximal axis (314) relative to proximal clevis (310); while movement of second cable (365) in a second, opposite direction pivots proximal pulleys (345) and distal clevis (305) about proximal axis (314) relative to proximal clevis (310). Proximal redirect pulley(s) (355) suitably guides second cable (365) along the predetermined path to thereby suitable engage proximal pulleys (345) in accordance with the description herein. Of course, in some instances, use of proximal redirect pulley (355) in relation to its engagement with second cable (360) is optional. Proximal pulleys (345) may include any other suitable features configured to suitably engage one or more cable(s) (365) as would be apparent to one skilled in the art in view of the teachings herein.

One or more proximal ends (not shown) of second cable (365) may be operatively coupled to suitable components such that when instrument (300) is suitably coupled to robotic arm (20, 32), instrument driver (24, 66) (or other suitable components) is configured to actuate second cable (365) in accordance with the description hearing. Therefore, when instrument (300) is operatively coupled to a robotic arm (20, 32) of robotic system (10, 28), robotic system (10, 28) may control the angular position of distal clevis (305), distal pulley (325), and end effector (315) about proximal axis (314) relative to proximal clevis (310).

Conductive cable (330) traverses a third path within the wrist (301) so as to engage with the proximal redirect pulleys (355), the proximal pulleys (345), and the distal pulley (325). A distal portion of conductive cable (330) is electrically coupled to the monopolar hook of end effector (315). Additionally, as best shown in FIG. 8, conductive cable (330) extends proximally from wrist (301) into electrical communication with a source (2) of monopolar energy (e.g., a suitable generator) configured to selectively produce and communicate monopolar energy along conductive cable (330). Such a source (2) of monopolar energy may be associated with a tower of robotic system (10, 28), with a portion of robotic arm (20, 32), or any other suitable component of robotic system (10, 28) as would be apparent to one skilled in the art in view of the teachings herein. Conductive cable (330) is configured to communicate therapeutic electrical energy from source (2) to monopolar hook of end effector (315) such that end effector (315) may therefore deliver therapeutic monopolar energy onto targeted tissue in contact with and/or suitably adjacent to end effector (315). Therefore, an operator may selectively activate end effector (315) with monopolar energy in order to suitably treat targeted tissue in accordance with the description herein.

During illustrative use, an operator may manipulate end effector (315) in order to suitably position end effector (315) adjacent to targeted tissue. The operator may manipulate end effector (315) utilizing suitable controls of robotic system (10, 28). Therefore, the operator may advance/retract instrument (300) and/or rotate instruments (300) about its own longitudinal axis in accordance with the description herein in order to suitably access and/or manipulate tissue. Additionally, or alternatively, the operator may utilize suitable controls of robotic system (10, 28) to pivot end effector (315) about pivots axes (312, 314) in order to suitably access and manipulate tissue. In some instances, end effector (315) may be further used to apply monopolar energy to targeted tissue via end effector (315) in accordance with the description herein.

Conductive cable (330) may be sufficiently flexible in order to suitably bend in response to articulation of wrist (301) in accordance with the description herein. Therefore, conductive cable (330) may bend into various positions in response to pivotal movement of end effector (315) and distal pulley (325) about distal pivot axis (312); as well as in response to pivotal movement of end effector (315), distal pulley (325), and distal clevis (305) about proximal pivot axis (314). It should be understood that conductive cable (330) is configured to suitably communicate electrical energy to end effector (315) while in the various bent positions during illustrative use.

Conductive cable (330) is electrically insulated from other components of instrument (300) and/or robotic system (10, 28) such that electrical energy generated from source (2) is communicated to end effector in a controlled manner. In other words, conductive cable (330) is sufficiently electrically insulated such that monopolar electrical energy is not inadvertently communicated to other components of instrument (300) besides end effector (315), nor is communicated to other portions of the external environment besides the targeted tissue.

### III. Illustrative Alternative Monopolar Hook

As mentioned above, while instrument (300) is suitably coupled with robotic system (10, 28), robotic system (10, 30) may advance/retract instrument (300), rotate instrument (300) about its own longitudinal axis, and/or pivot end effector (315) about distal pivot axis (312) and/or proximal pivot axis (314). In some instances, end effector (315) may encounter exposure to high forces/stresses/loads, which may then be distributed to distal pulley (315). For example, robotic system (10, 28) may have multiple robotic arms (20, 32), each controlling a respective instrument. In such instances, end effector (315) may inadvertently collide with other robotic tools, thereby generating high forces/stresses/loads onto end effector (315). Exposure to such high forces/stresses/loads may lead to undesirable deformation, or even failure, of end effector (315) and/or distal pulley (315). Deformation may cause end effector (315) to no longer be substantially fixed to distal pulley (315) (e.g., end effector (315) may associate with distal pulley (315) but is loosely attached with an undesirable degree of slop, play, lash, backlash, clearance, etc.), which may undesirably affect the operator's ability to control end effector (315). Failure may cause pieces of end effector (315) and or distal pulley (315) to undesirably disassociate from the rest of instrument (300). Therefore, it may be desirable to have a monopolar hook configured to withstand such high forces/stresses/loads while inhibiting an undesirable amount of dissociation between end effector (3150) and distal pulley (315). Further it may be desirable to control the location of deformation to inhibit failure (e.g., components breaking apart into multiple pieces).

As also mentioned above, conductive cable (330) is electrically coupled to monopolar hook of end effector (315) such that conductive cable (330) is configured to communicate therapeutic electrical energy from source (2) to monopolar hook of end effector (315). It may be desirable to electrically insulate conductive cable (330) from other components of instrument (300) and/or robotic system (10, 28) (besides end effector (315) and source (2)) such that electrical energy generated from source (2) is communicated to end effector (315) in a controlled manner. In other words, it may be desirable to ensure conductive cable (330) is sufficiently electrically insulated such that monopolar electrical energy is not inadvertently communicated to other components of instrument (300) besides end effector (315), nor is communicated to other portions of the external environment besides the targeted tissue. Further, in some instances, it may be desirable to provide a reliable means of applying a dielectric material between conductive cable (330) and portions of distal pulley (325) housing conductive cable (330). Further, in some instances, during the manufacture of end effector (315) and distal pulley (325), it may be desirable to provide a reliable means of electrically coupling the distal end of conductive cable (330) with monopolar hook of end effector (315).

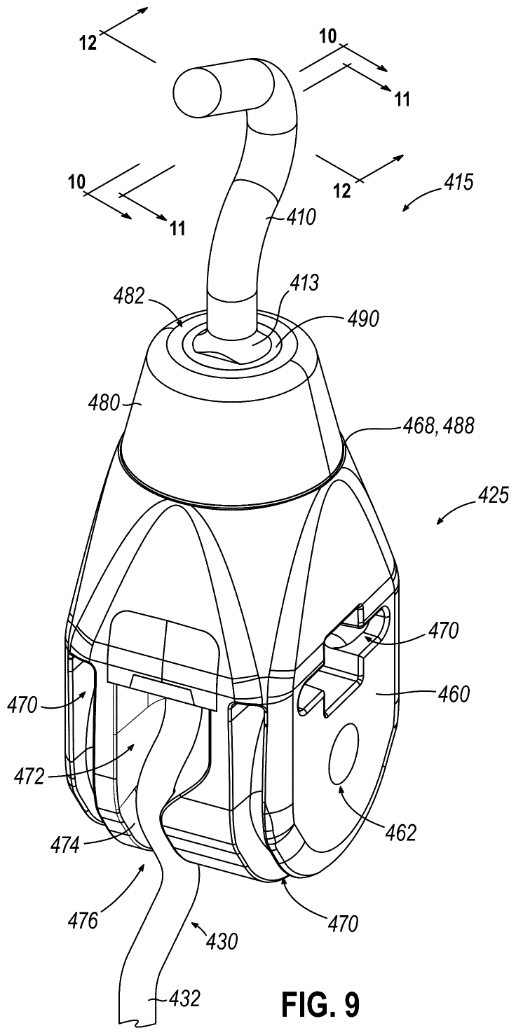

FIGS. 9-12 show an illustrative end effector (415), an illustrative distal pulley assembly (425), and an illustrative conductive cable (430) that may be readily incorporated into instrument (300) described herein in replacement of end effector (315), distal pulley (325), and conductive cable (330), respectively. End effector (415), distal pulley assembly (425), and conductive cable (430) may be substantially similar to end effector (315), distal pulley (325), and conductive cable (330) described above, respectively, with differences elaborated below.

Therefore, distal pulley assembly (425) is configured to pivotally couple with distal clevis (305) such that distal clevis (305), distal pulley assembly (425), and end effector (415) may pivot about proximal pivot axis (314) in accordance with the description above; and such that distal pulley assembly (415) and end effector (425) may pivot about distal pivot axis (312) in accordance with the description above. Further, distal pulley assembly (425) is configured to operatively couple with one or more cables (360) to allow for robotic system (10, 28) to suitably control the pivotal position of distal pulley assembly (425) and end effector (415) relative to distal clevis (305) in accordance with the description herein. Additionally, conductive cable (430) traverses a path proximally away from distal pulley assembly (425) in order to electrically couple end effector (415) with a suitable electrical power source (2) such that robotic system (10, 28) may selectively activate end effector (415) with therapeutic monopolar energy in accordance with the teachings herein.

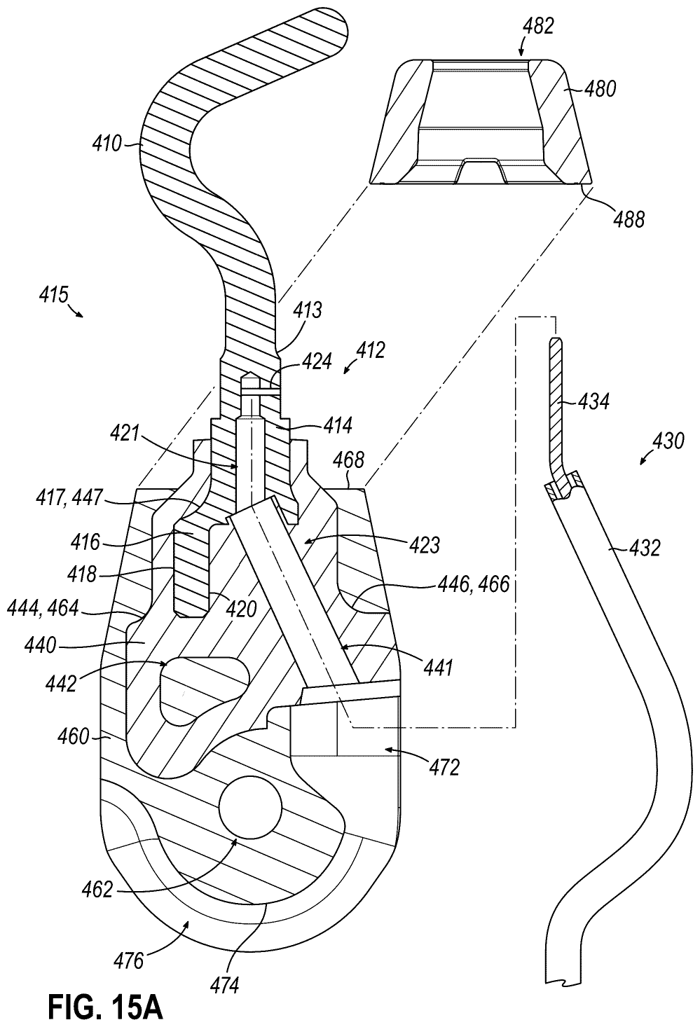

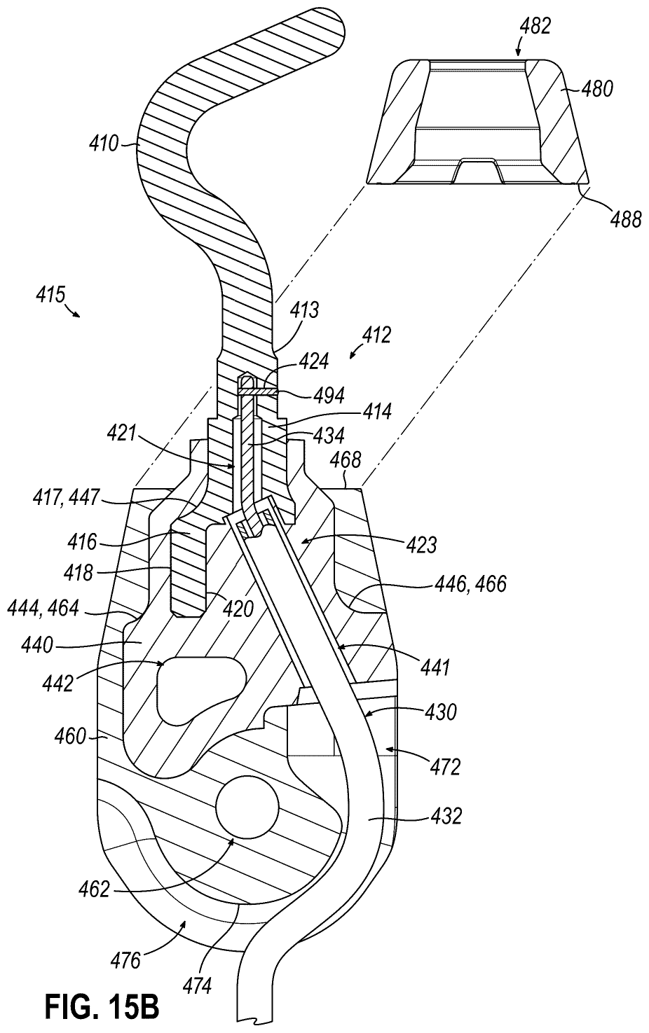

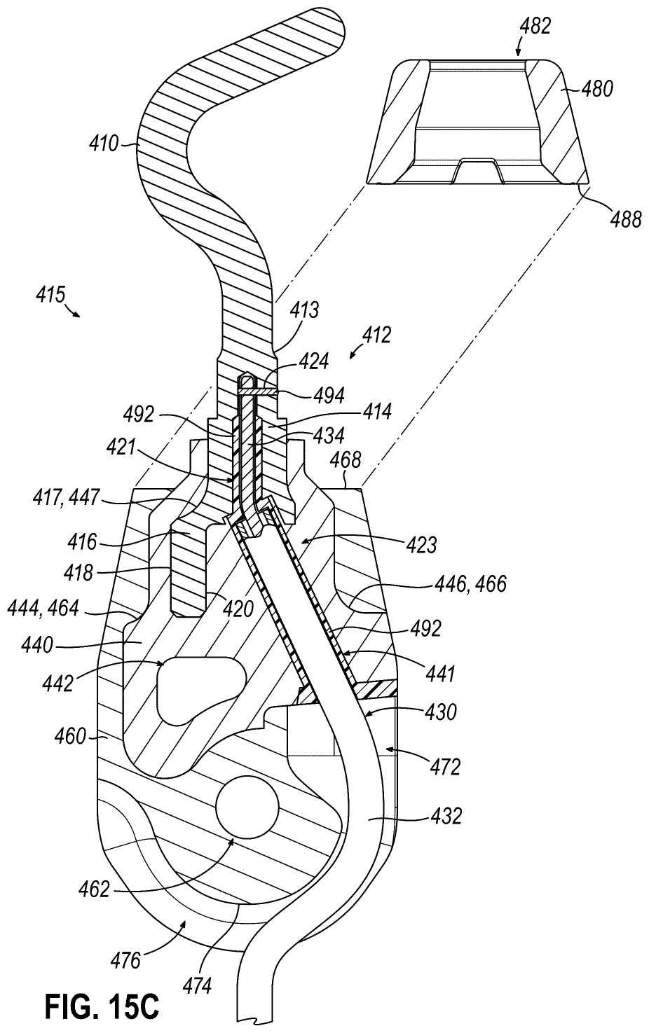

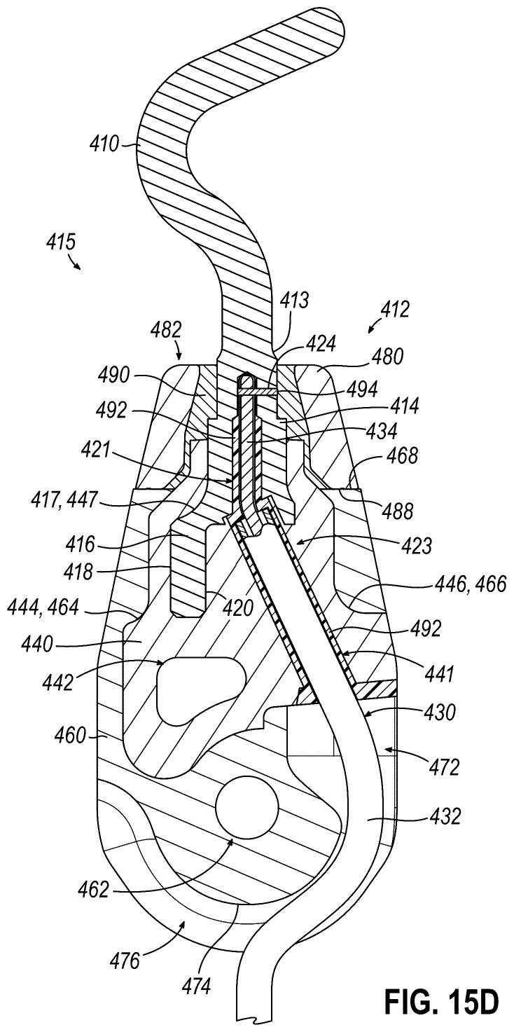

As best shown in FIG. 15A, conductive cable (430) includes an insulating outer jacket (432) and a conductive core wire (434). Insulating outer jacker (432) houses suitable portions of conductive core wire (434) in order to electrically isolate core wire (434) from components other than monopolar energy source (2) and end effector (415). Conductive core wire (434) is configured to communicate therapeutic monopolar energy from energy source (2) to end effector (415). A distal portion of conductive core wire (434) extends distally from the terminating distal end of outer jacket (432). The exposed distal portion of conductive core wire (434) is dimensioned to be housed within the confines of end effector (415) in order to establish electrical communication with end effector (415) in accordance with the description herein.

As will be described in greater detail below, end effector (415) and distal pulley assembly (425) have various features designed to enable end effector (415) to suitably transmit inadvertent large forces/stresses/loads experienced by end effector (415) onto distal pulley assembly (425), thereby inhibiting end effector (415) from becoming loosely attached to distal pulley assembly (425) with an undesirable degree of slop, play, lash, backlash, clearance, etc. Further, as will be described in greater detail below, end effector (415) and distal pulley assembly (425) have various features that promote effectively electrically coupling end effector (415) with conductive cable (430), and/or promote electrical isolation of conductive cable (430) from components other than end effector (415) and/or inhibiting damage to conductive cable (430).

Turning back to FIGS. 9-12, end effector (415) of the current example includes a distal hook (410) that extends proximally into a cable receiving collar (414) via a curved surface (413). Distal hook (410) may be substantially similar to the distal hook of end effector (315) described above, with differences elaborated herein. Proximal receiving collar (414) terminates proximally into a laterally expanded base (416) having an enlarged laterally and upwardly facing surface area. Distal hook (410), cable receiving collar (414), and base (416) may be formed from a single piece of material. Various geometric shapes of end effector (415) may be formed via suitable machining processes and techniques as would be apparent to one skilled in the art in view of the teachings herein.

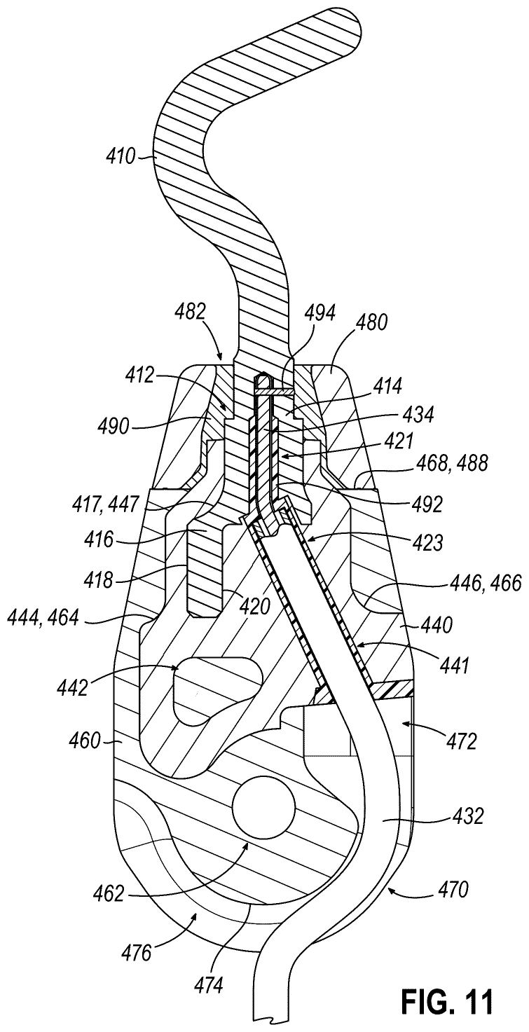

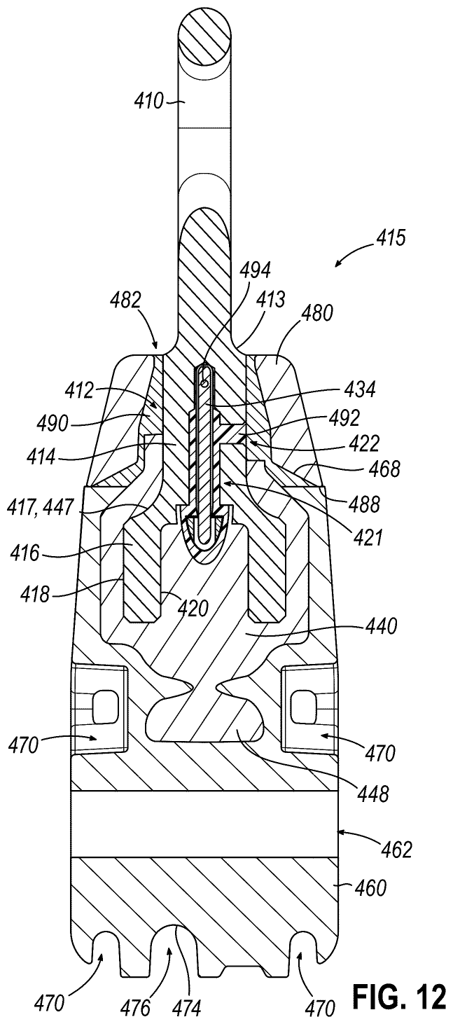

As best shown in FIGS. 11-12, proximal cable receiving collar (414) defines a conductive wire channel (421). Conductive wire channel (421) is dimensioned to suitably receive the distal portion of conductive core wire (434) exposed from insulating jacket (432). Conductive wire channel (421) terminates distally within cable receiving collar (414) such that a distal end of conductive core wire (434) may be inserted adjacent to the distal end of conductive wire channel (421) while remaining within the confines of cable receiving collar (414).

As best shown in FIGS. 11, 13A, and 14A, proximal cable receiving collar (414) defines a coupling port, such as welding port (424). Welding port (424) extends between an external surface of cable receiving collar (414) and the interior surface defining conductive wire channel (421). Welding port (424) provides suitable access for a manufacturer to weld (or otherwise suitably couple) a distal portion of conductive core wire (434) with end effector (415). For instance, as will be described in greater detail below, a manufacturer may insert the distal portion of core wire (432) into conductive wire channel (421) until a portion of core wire (432) is suitably aligned with a portion of conductive wire channel (421) directly adjacent to welding port (424). A manufacturer may visually confirm suitable placement of core wire (432) within conductive wire channel (421) by viewing the presence of core wire (432) within wire channel (421) via welding port (424). Once core wire (432) is suitably positioned in accordance with the description herein, a manufacturer may utilize a penetrative weld (494) to couple core wire (434) to the interior surface of cable receiving collar (414) via access provided by welding port (424). Such a penetrative weld (494) may be formed my melting suitable portions of core wire (434) and collar (414) (or any other suitable portion of end effector (415)) and fusing them together by use of an industrial laser. Such a penetrative weld (494) may ensure a robust (e.g., strong with low resistance) electrical connection. Therefore, welding port (424) may provide both visual access and welding access during the coupling of core wire (434) and end effector (415) in accordance with description herein.

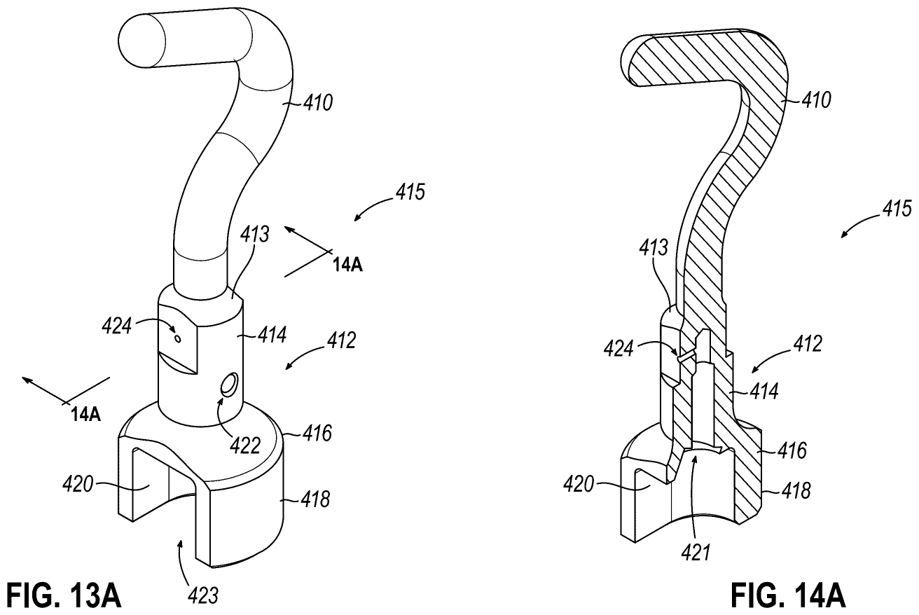

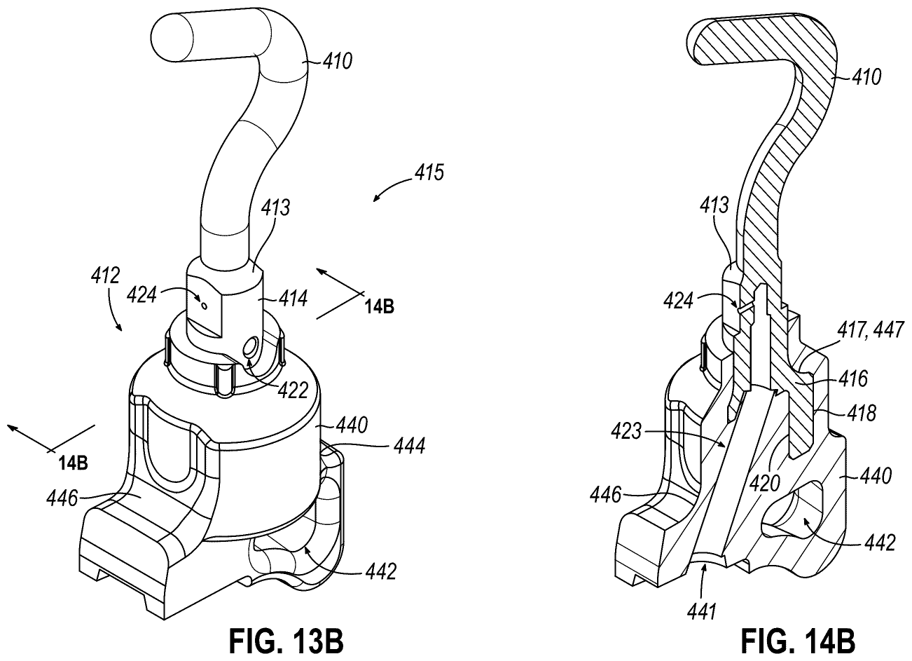

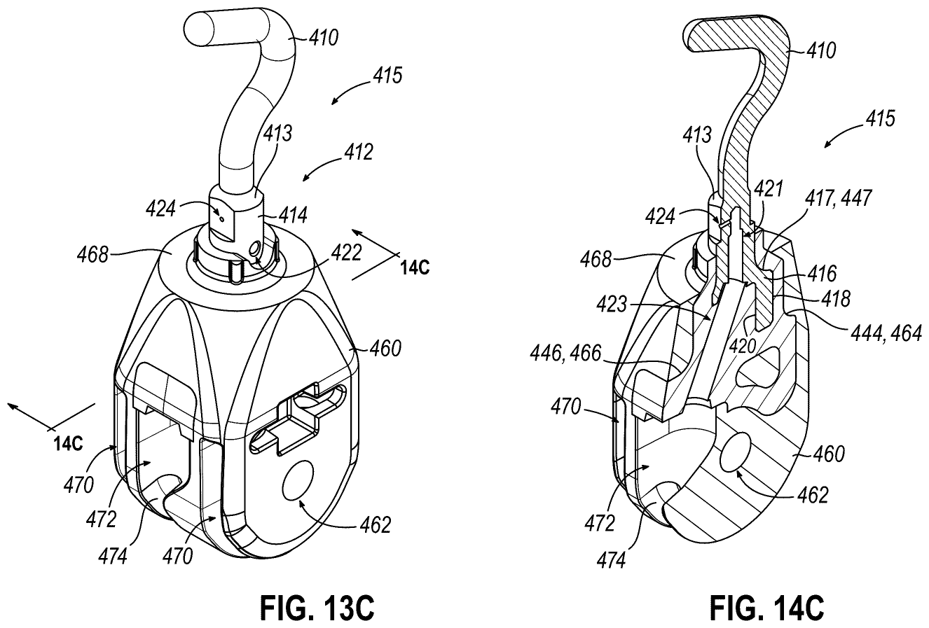

Additionally, prior to coupling of core wire (432) with end effector (415), welding port (424) may be utilized for other suitable purposes as would be apparent to one skilled in the art in view of the teachings herein. For example, after end effector (415) has been suitably manufactured as shown in FIGS. 13A and 14A, welding port (424) may be utilized as a flushing port. For example, pressurized fluid may be distributed into conductive wire channel (421) via welding port (424) in order to suitably remove any undesirable objects/byproduct/debris/housed within wire channel (421). Therefore, welding port (424) may be utilized in order to suitably clear the interior of wire channel (421) during suitable moments of the manufacturing process of end effector (415) and/or distal pulley assembly (425) as would be apparent to one skilled in the art in view of the teachings herein.

Additionally, at least a portion of conductive wire channel (421) is large enough to receive conductive core wire (434) as well as a dielectric cable sealant (492) in accordance with the description herein. Dielectric cable sealant (492) is configured to electrically isolate the distal exposed portion of core wire (434) during illustrative use. As will be described in greater detail below, cable receiving collar (414) includes a sealant port (422) (see FIG. 13A) that allows for the uniform distribution/delivery of dielectric cable sealant (492) after end effector (415) has been suitably coupled to core wire (434) of conductive cable (430) in accordance with the description herein.

As best shown in FIGS. 13A and 14A, a proximal portion of conductive wire channel (421) is also defined by base (416). Base (416) also defines a laterally presented window (423) that is in communication with conductive wire channel (421). Window (423) and conductive wire channel (421) are in suitable communication with each other such that suitable portions of conductive cable (430) may extend proximally away from conductive wire channel (421) via window (423) and into a cable pathway (441) defined by inner body (440) of distal pulley assembly (425).

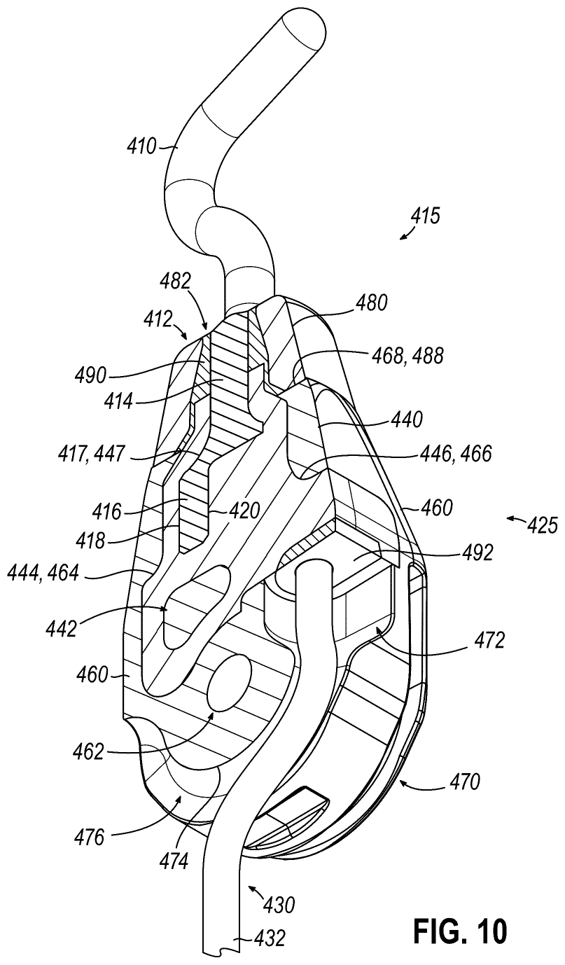

Base (416) of the current example includes a semi-cylindrical shell that has a larger lateral dimension compared to cable receiving collar (414), as well as larger laterally facing surfaces and upward facing surface(s). The semi-cylindrical shell of base (416) includes a laterally facing exterior surface (418) and a laterally facing interior surface (420). As best shown in FIGS. 10-12, a top portion of base (416) includes a scalloped surface (417). During the manufacture of distal pulley assembly (425), as shown in FIGS. 13B and 14B, inner body (440) of distal pulley assembly (425) is configured to be injection molded onto base (416) such that base (416) and a proximal portion of cable receiving collar (414) are housed within inner body (440) and in direct contact with interior surfaces of inner body (440). Such molding allows the formation of complementary interior surfaces of inner body (440) with regards corresponding surfaces of base (416) and cable receiving collar (414).

Scalloped surface (417) of base (416) is directly in contact with a complementary scalloped surface (447) of inner body (440); while exterior surface (418) and interior surface (420) of base are also directly in contact with complementary surfaces of inner body (440). Injection molding of inner body (440) onto base (416) and proximal portion of cable receiving collar (414) may allow for uniform (or substantially uniform) contact between such complementary surfaces. Of course, any other suitable manufacturing technique allowing for uniform (or substantially uniform) contact between complementary surface may be utilized as would be apparent to one skilled in the art in view of the teachings herein.

The larger lateral and upward facing surfaces (418, 420, 417) of base (416) (as compared to cable receiving collar (414) and/or hook (410)) allow for exterior surface (418) and interior surface (420) to have large surface areas compared to collar (414) and/or hook (410). The uniform contact between base (416) and corresponding complementary surfaces of interior body (440) allows for a large surface area of load distribution from base (416) onto inner body (440). In other words, the large surface areas of exterior and interior surfaces (418, 420) and/or scalloped surface (417) may allow end effector (415) to distribute large forces onto interior body (440) with a reduced stress profile, thereby inhibiting the chances of plastic deformation of inner body (440) when end effector (415) experiences such large forces/loads/stresses in accordance with the description herein. This may help ensure that when end effector (415) experiences such large forces/loads/stresses, end effector (415) and inner body (440) remain substantially fixed relative to each other afterwards (i.e., inhibit the chances that end effector (415) becomes loosely attached with distal pulley assembly (415) with an undesirable degree of slop, play, lash, backlash, clearance, etc.).

Additionally, the curved geometric profile between scalloped surface (417) of base (416) and complementary scalloped surface (447) of inner body (440) may also help ensure that when end effector (415) experiences such large forces/loads/stresses, end effector (415) and inner body (440) remain substantially fixed relative to each other afterwards (i.e., inhibit the chances that end effector (415) becomes loosely attached with distal pulley assembly (415) with an undesirable degree of slop, play, lash, backlash, clearance, etc.).

As mentioned above, distal hook (410) extends proximally into a cable receiving collar (414) via a curved surface (413). Curved surface (413) extends between distal hook (410) and collar (414) such that distal hook (410) has a smaller lateral profile. In other words, the lateral thickness between opposing exterior surfaces of collar (414) is greater than the lateral thickness between opposing exterior surfaces of hook (410) when viewed from the perspective shown in FIGS. 11 and 12. Curved surface (413) is dimensioned such that when end effector (415) experiences large forces/loaded/stress that will elastically and/or plastically deform end effector (415), such deformation occurs at or adjacent to curved surface (413) interposed between hook (410) and cable receiving collar (414). As also shown in FIGS. 11 and 13, curved surface (413) is located distally relative to a distal end of distal pulley assembly (425). The deformation of end effector (415) at curved surface (413) may act as a failsafe to help ensure that other components of end effector (415) and/or distal pulley assembly (425) do not break and/or fail. Therefore, in instances where end effector (415) encounters deforming force/load/stress, curved surface (413) inhibits breaking/failure of components that may inadvertently and undesirably be released into the surgical environment.

Distal pulley assembly (425) includes a first injection molded body or inner body (440), a second injection molded body or outer body (460), and a ceramic cap (480). Inner body (440) and outer body (460) may be formed of any suitable material as would be apparent to one skilled in the art in view of the teachings herein. Bodies (440, 460) may be formed of a material that is electrically insulative in nature, thereby inhibiting therapeutic monopolar energy from being inadvertently communicated through bodies (440, 460). While a ceramic cap (480) is used in the current example, cap (480) may be formed of any suitable material as would be apparent to one skilled in the art in view of the teachings herein.

As will be described in greater detail below, inner body (440) defines the portions of distal pulley assembly (425) configured to receive and mate with conductive cable (430), while other body (460) includes the various features utilized to operatively couple distal pulley assembly (425) with the other components of instrument (300). Further, as will be described in greater detail below, ceramic cap (480) is configured isolate the distal hook portion (410), both thermally and electrically, from the remaining components of distal pulley assembly (425); while also inhibiting significant load transfers from hook portion (410) onto ceramic cap (480) itself.

As mentioned above, and as shown in FIGS. 13B and 14B, inner body (440) may be formed by being injection molded onto suitable surfaces of cable receiving collar (414) and base (416) of end effector (415). After inner body (440) is formed, as shown in FIGS. 13B and 14B, ports (422, 424) defined by cable receiving collar (414) are still accessible. Any suitable techniques, methods, and equipment may be utilized in order to suitably injection mold inner body (440) onto suitable portions of end effector (415) as would be apparent to one skilled in the art in view of the teachings herein. After inner body (440) is injection molded onto cable receiving collar (414) and base (416) of end effector (415), engagement/contact between complementary surfaces of inner body (440) and end effector (415) substantially fixes inner body (440) relative to end effector (415).

As best shown in FIG. 12, portions of inner body (440) extending laterally outward (e.g., left to right from the viewpoint shown in FIG. 12) from exterior surface (418) of base (416) have a lateral thickness that is suitably thick enough in order to structurally support inner body (440) in instances where end effector (415) encounters a large force/load/stress in accordance with the description herein, yet thin enough to maintain a suitable overall thickness of distal pulley assembly (425) for use in accordance with the description herein. Therefore, portions of inner body (440) directly adjacent to exterior surface (418) of base (416) have a large enough lateral thickness (when viewed from the perspective shown in FIG. 12) such that those portions of inner body (440) may receive forces/loads/stresses from base (416) of end effector (415) without undesirably deforming, breaking, etc.

As mentioned above, complementary scalloped surface (447) suitably engages scalloped surface (417) of base (416) in order to promote suitable engagement/contact between inner body (440) and end effector (415) in accordance with the description herein. Additionally, as best shown in FIGS. 10-11 and 13B, inner body (440) also defines through hole (442) and includes a two protrusions having a respective scalloped surface (444, 446). Additionally, as best shown in FIG. 12, inner body (440) also includes a proximal dovetail (448). During the manufacture of distal pulley assembly (425), as shown in FIGS. 13C and 14C, outer body (460) of distal pulley assembly (425) is configured to be injection molded onto inner body (440) such a proximal portion of inner body (440) is housed within outer body (446) and in direct contact with interior surfaces of outer body (460). Such molding allows the formation of complementary interior surfaces of outer body (460) with regards corresponding surfaces of inner body (440).

Scalloped surfaces (444, 446) of inner body (440) are directly in contact with a corresponding complementary scalloped surface (464, 466) of outer body (460). Outer body (460) fills the interior defined by though hole (442) such that complementary surfaces of outer body (460) are in direct contact with the surfaces of inner body (440) defining through hole (442). Further, suitable portions of outer body (460) are also in direct contact with outer surfaces of dove tail (448). Injection molding of outer body (460) onto inner body (440) may allow for uniform (or substantially uniform) contact between such complementary surfaces. Of course, any other suitable manufacturing technique allowing for uniform (or substantially uniform) contact between complementary surfaces may be used as would be apparent to one skilled in the art in view of the teachings herein.

The uniform contact between suitable portions of inner body (440) (e.g., scalloped surfaces (444, 446), through hole (442), and dovetail (448)) and corresponding complementary surfaces of outer body (460), as well as the geometric profiles of such surfaces, allow for inner body (440) to transmit large loads/forces/stress onto outer body (460) while also inhibiting the chances of plastic deformation of both inner body (440) and outer body (460) when end effector (415) experiences such large forces/loads/stresses in accordance with the description herein. This may help ensure that when end effector (415) experiences such large forces/loads/stresses, inner body (440) and outer body (460) remain substantially fixed relative to each other afterwards (i.e., inhibit the chances that inner body (440) becomes loosely attached with outer body (460) with an undesirable degree of slop, play, lash, backlash, clearance, etc.).

As best shown in FIGS. 11 and 14B, inner body (440) defines a cable pathway (441) that is in communication with conductive wire channel (421) defined by cable receiving collar (414) and base (416) of end effector (415). Inner body (440) may help electrically isolate the portions of end effector (415) housed by inner body (440).

In the current illustrative example, cable pathway (441) extends distally from proximal end of inner body (440) at an oblique angle relative to the longitudinal axis of inner body (440). The proximal end of cable pathway (441) is in communication with a cable port (472) (see FIGS. 13C and 14C) defined by outer body (460) of distal pulley assembly (425). Further, cable pathway (441) extends into the interior of base (416) in order to communicate with conductive wire channel (421) at a proximal end of cable receiving collar (414). Therefore, when assembled, cable pathway (441) is dimensioned to house a portion of conductive cable (430). Additionally, cable pathway (441) is large enough to receive conductive cable (430) as well as a dielectric cable sealant (492) such that the dielectric cable sealant (492) is located on an external surface of cable (430). When fully assembled, as best shown in FIG. 11, dielectric cable sealant (492) extends around suitable portions of cable (430) from the proximal end of cable pathway (441) all the way into at least a portion of conductive wire channel (421). Dielectric cable sealant (492) is configured to promote electrical isolation of the exposed distal portion of core wire (434) such that therapeutic monopolar energy emitted by the exposed distal portion of core wire (434) during illustrative use in accordance with the description herein is inhibited from reaching the external environment via cable port (472). The longer the length which cable sealant (492) extends, the greater the electrically insulative properties of cable sealant (492). Therefore, the length of cable pathway (441) helps improve the electrical insulating properties of cable sealant (492).

As best shown in FIGS. 15A-15B, after outer body (460) is formed onto inner body (440) in accordance with the description herein, conductive cable (430) may be inserted into cable pathway (441) and conductive wire channel (421) via cable port (472) in order to suitably couple cable (430) with end effector (415). Cable pathway (441) extends along a substantially linear path, thereby allowing for easy initial insertion of cable (430) into cable pathway (441) via port (472). In particular, a manufacturer may simply insert a distal end of cable (430) into pathway (441) via port (472) and further insert cable (430) until distal end (430) is suitably seated within conductive wire channel (421).

Further, cable (430) may be initially inserted and attached onto distal pulley assembly (425) after the formation of outer body (460). Attaching cable (430) onto distal pulley assembly (425) after the formation of outer body (460), rather than prior to the formation of outer body (460), may reduce the chances of cable (430) becoming damaged during manufacture. For instance, if cable (430) were attached prior to the formation of outer body (460), cable (430) may become inadvertently severed/damaged while outer body (460) is formed.

As mentioned above, and as shown in FIGS. 13C and 14C, outer body (460) may be formed by being injection molded onto suitable surface of inner body (440). After outer body (460) is formed, ports (422, 424) defined by cable receiving collar (414) are still accessible. As will be described in greater detail below, providing access to ports (422, 424) after formation of outer body (460) may allow a manufacturer to suitably distribute dielectric cable sealant (492) and apply a penetrative weld (494) in accordance with the description herein, after the formation of outer body (460). Any suitable techniques, methods, and equipment may be utilized in order to suitably injection mold outer body (460) onto suitable portions of inner body (440) as would be apparent to one skilled in the art in view of the teachings herein. After outer body (460) is injection molded onto inner body (440), engagement/contact between complementary surfaces of inner body (440) and outer body (460) substantially fixes inner body (440) relative to outer body (460).

As mentioned above, complementary scalloped surfaces (464, 466) and other portions of outer body (460) engaged with through hole (442) and dove tail (448) promote suitable engagement/contact between inner body (440) and outer body (460) in accordance with the description herein. As best shown in FIG. 12, portions of outer body (450) directly adjacent to inner body (460) and exterior surface (418) of base (416) have a lateral thickness that is suitably thick enough in order to structurally support outer body (460) in instances where end effector (415) encounters a large force/load/stress in accordance with the description herein, yet thin enough to maintain a suitable overall thickness of distal pulley assembly (425) for use in accordance with the description herein. Therefore, portions of outer body (460) adjacent to exterior surface (418) of base (416) have a large enough lateral thickness (when viewed from the perspective shown in FIG. 12) such that those portions of outer body (460) may receive forces/loads/stresses from inner body (440) of pulley assembly (425) and base (416) of end effector (415) without undesirably deforming, breaking, etc.

As mentioned above, outer body (460) includes features utilized to operatively couple distal pulley assembly (425) with the other components of instrument (300). Outer body (460) extends proximally relative to inner body (440). As best shown in FIG. 9, outer body defines a pivot axis through hole (462), a pair of pivot cable openings (470), cable port (472), and a cable housing (470).

Pivot axis through hole (462) is dimensioned to receive a pivot axis, such as distal axel (320) described above, in order to pivotally couple distal pulley assembly (425) and end effector (415) to distal clevis (305) of instrument (300). Each pivot cable opening (470) is dimensioned to receive a cable, such as cable (360) described above, in order to allow robotic system (10, 28) to operatively pivot distal pulley assembly (425) and end effector (415) about the pivot axis defined by pivot axis through hole (462) in accordance with the description herein. Outer body (460) may have, or be configured to couple to, any other suitable structures in order to operatively couple distal pulley assembly (425) to suitable components of instrument (300) as would be apparent to one skilled in the art in view of the teachings herein.

Outer body (460) also include a cable engagement surface (474) that defines cable housing (476). Cable engagement surface (474) is configured to suitably contact portions of cable (430) (e.g., receive or release) as distal pulley assembly (425) pivots about the axis defined by pivot axis through hole (462). Therefore, cable engagement surface (474) may selectively bend cable (430) as distal pulley assembly (425) pivots in accordance with the description herein. As best shown in FIG. 10, cable engagement surface (474) has a sufficiently large bend radius in order to inhibit cable (430) from kinking (or otherwise being damaged) in response to pivotal movement of distal pulley assembly (425). Therefore, the large bend radius of cable engagement surface (474) may help increase the life span of cable (430) from cyclical fatigue associated with pivotal movement of distal pulley assembly (425).

Turning to FIGS. 10-12 and 15A-15D, outer body (460) also includes a cap engagement surface (468). Cap engagement surface (468) is configured to contact proximally presented annular shoulder (488) of ceramic cap (480) in order to suitably receive ceramic cap (480) during the manufacture of distal pulley assembly (425). Therefore engagement between cap engagement surface (468) and proximally presented annular shoulder (488) of ceramic cap (480) may promote locating ceramic cap (480) relative to the rest of distal pulley assembly (425) during assembly of ceramic cap (480) in accordance with the description herein.

As best shown in FIGS. 10-12, ceramic cap (480) defines an interior (482) extending from a proximal end to a distal end of cap (480). Interior (482) is dimensioned to house cable receiving collar (414) of end effector (415). When assembled, cap sealant (490) is suitably interposed between the interior surface of ceramic cap (480) and the exterior surface of cable receiving collar (414). Therefore, interior (482) is suitably large enough to remain mechanically isolated from cable receiving collar (414) of end effector (415) via cap sealant (490). Additionally, ceramic cap (480) is configured to electrically and thermally isolate hook portion (410) from the rest of distal pulley assembly (425). This may make cleaning eschar off end effector (415) and distal pulley assembly (425) easier.

End effector (415) and cap (480) are suitably isolated from each over via the cap sealant (490) such that when end effector (415) experiences a high load/force/stress in accordance with the description herein, such a load/force/stress is not significantly transferred from end effector (415) onto cap (480). Therefore, cap sealant (490) may at least partially absorb the mechanical loads imparted on end effector (415). Inhibiting the significant transfer of such loads may also decrease the chances of ceramic cap (480) failing (e.g., breaking into multiple pieces) when end effector (415) encounters a high load/force/stress in accordance with the description herein. Cap Sealant (490) may be formed of any suitable material as would be apparent to one skilled in the art in view of the teachings herein. As one illustrative example, cap sealant (490) may include a silicone material.

FIGS. 13A-15D show an illustrative manufacturing process of end effector (415) and distal pulley assembly (425). First, as shown in FIGS. 13A and 14A, end effector (415) may be manufactured in accordance with the description herein. As mentioned above, welding port (424) may be utilized in order to distribute fluid into conductive wire channel (421) to thereby remove undesirable debris and/or manufacturing byproduct from within the confines of conductive wire channel (421). Next, as shown in FIGS. 13B and 14B, inner body (440) of distal pulley assembly (425) may be injection molded onto the surfaces of cable receiving collar (414) and base (416) of end effector (415) such that inner body (440) is substantially fixed relative to end effector (415). Next, as shown in FIGS. 13C and 14C, outer body (460) of distal pulley assembly (425) may be injection molded onto the surfaces of inner body (440), such that outer body (460) is substantially fixed to inner body (440).

Once outer body (460) and inner body (440) are both formed onto end effector (415), cable (430) may be suitably coupled to end effector (415). Next, as shown in FIGS. 15A-15B, a manufacturer may obtain cable (430) insert the distal exposed portion of core wire (434) into cable pathway (441) and conductive wire channel (421) via cable port (472) of outer body (460). The manufacture may utilize weld port (424) to visually confirm that core wire (434) is suitably positioned within conductive wire channel (421) in preparation of being electrically coupled with end effector (415). Next, as also shown in FIG. 15B, a manufacturer may couple core wire (434) with end effector (415) via a penetrative weld (494) via weld port (424).

Next, as shown in FIG. 15C, once core wire (434) is attached to end effector (415) via penetrative weld (494), dielectric cable sealant (492) may be applied within conductive wire channel (421) and cable pathway (441) around the corresponding exterior surfaces of conductive cable (430). As mentioned above, cable receiving collar (414) includes a sealant port (422) (see FIG. 13A) that allows for the uniform distribution/delivery of dielectric cable sealant (492). Therefore, a manufacturer may insert a suitable delivery nozzle (or other delivery source) configured to deliver dielectric cable sealant (492) within sealant port (422). With the deliver nozzle located within sealant port (422), the manufacture may inject or otherwise deliver dielectric cable sealant (492) within conductive wire channel (421) and cable pathway (441) until dielectric cable sealant (492) suitably fills wire channel (421) and cable pathway (441). Previously, without sealant port (422), a manufacturer may have been required to pre-apply dielectric cable sealant (492) onto the outer surfaces of cable (430) prior to inserting cable (430) within end effector (415) and distal pulley assembly (425) in accordance with the description herein. Such application of dielectric cable sealant (492) may lead to inconsistent application of sealant (492) on cable (430), which may undesirably affect the quality of insulation capabilities of cable sealant (492). Therefore, sealant port (422) may be utilized in order to consistently apply cable sealant (492) in accordance with the description herein.

Next, as shown in FIG. 15D, after cable sealant (492) is suitably applied, a manufacturer may apply cap sealant (490) onto a top portion of outer body (460) in order to suitably cover cable receiving collar (414) and a top portion of inner body (440). Once cap sealant (490) is applied, the manufacturer may insert ceramic cap (480) over distal hook portion (410) until proximally presented annular shoulder (488) is suitably located against cap engagement surface (468) of outer body (460).

### IV. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a conductive cable; (b) a pulley assembly, the pulley assembly comprising: (i) an inner body defining a cable pathway extending between a proximal end and a distal end, (ii) an outer body fixedly attached to the inner body, the outer body defining a cable port in communication with the proximal end of the cable pathway, and (iii) a distal cap defining an interior; and (c) an end effector electrically coupled to the conductive cable, comprising: (i) a distal hook configured to receive therapeutic energy from the conductive cable and deliver the received therapy energy to tissue, (ii) a proximal body fixed relative to the distal hook, wherein the proximal body defines a conductive wire channel in communication with the distal end of the cable pathway defined by the inner body, wherein the conductive cable extends through the cable port of the outer body, the cable pathway of the inner body, and into the conductive wire channel, wherein the proximal body defines a first port located within the interior of the distal cap, wherein the first port extends from an outer surface of the proximal body into the conductive wire channel.

### Example 2

The apparatus of Example 1, wherein the proximal body defines a second port located within the interior of the distal cap, wherein the second port extends from the outer surface of the proximal body into the conductive wire channel.

### Example 3

The apparatus of Example 2, wherein the second port is located proximally relative to the first port.

### Example 4

The apparatus of any one or more of Examples 1-3, further comprising a cap sealant located within the interior of the distal cap, wherein the cap sealant is interposed between the distal cap and the outer surface of the proximal body located within the interior.

### Example 5

The apparatus of Example 4, wherein the cap sealant mechanically isolates the distal cap from the end effector.

### Example 6

The apparatus of any one or more of Examples 1 through 5, further comprising a penetrating weld located within the first port, wherein the penetrating weld electrically couples the conductive cable with the end effector.

### Example 7

The apparatus of any one or more of Examples 1 through 6, further comprising a dielectric cable sealant located within the cable pathway of the inner body and the conductive wire channel.

### Example 8

The apparatus of Example 7, wherein the dielectric cable sealant is located within the first port.

### Example 9

The apparatus of either Example 7 or 8, wherein the dielectric cable sealant extends from the proximal end to the distal end of the cable pathway.

### Example 10

The apparatus of any one or more of Examples 1-9, wherein the proximal body of the end effector further comprises a base defining a portion of the conductive wire channel, wherein the base comprises a semi-cylindrical shell having an interior surface and an exterior surface, wherein the inner body is directly attached to both the interior surface and the exterior surface of the base.

### Example 11

The apparatus of Example 10, wherein the base defines a window, wherein a portion of the cable pathway extends through the window.

### Example 12

The apparatus of any one or more of Examples 1 through 11, wherein the proximal body of the end effector comprises a scalloped surface, wherein the inner body of the pulley assembly comprises a complementary scalloped surfaced directly engaged with the scalloped surface of the end effector.

### Example 13

The apparatus of any one or more of Examples 1 through 11, wherein the inner body defines a though hole, wherein the outer body comprises a section extending within the through hole and directly engaging a portion of the inner body defining the through hole.

### Example 14

The apparatus of any one or more of Examples 1 through 11, wherein the inner body comprises a proximal dovetail, wherein the outer body comprises a section directly engaged with the dovetail of the inner body.

### Example 15

The apparatus of any one or more of Examples 1 through 14, wherein the inner body comprises a scalloped surface, wherein the outer body comprises a section directly engaged with the scalloped surface of the inner body.

### Example 16

An apparatus, comprising: (a) a conductive cable; (b) a pulley defining a cable pathway housing a portion of the conductive cable; and (c) an end effector electrically coupled to the conductive cable, where in the end effector comprises: (i) a distal hook portion extending distally from the pulley, wherein the distal hook portion is configured to receive therapeutic energy from the conductive cable and deliver the received therapy energy to tissue, wherein the distal hook portion comprises a first lateral thickness, (ii) a proximal body attached to the pulley, wherein the proximal body comprises a second lateral thickness, wherein the second lateral thickness is greater than the first lateral thickness, and (iii) a curved surface located between, and coupling together, the distal hook portion and the proximal body, wherein the curved surface is configured to define a controlled yield location.

### Example 17

The apparatus of Example 16, wherein the curved surface forms an annular surface

### Example 18

The apparatus of either Example 16 or 17, wherein the pulley comprises a conducive cable contact surface.

### Example 19

A method of manufacturing a monopolar hook and a pulley assembly, the method comprising: (a) injection molding an inner body of the pulley assembly onto a proximal body of the monopolar hook; (b) injection molding an outer body of the pulley assembly onto the inner body of the pulley assembly; and (c) after injection molding the outer body of the pulley assembly, inserting a conductive cable within an interior of the monopolar hook and welding the conductive cable with the monopolar hook.

### Example 20

The method of Example 19, wherein inserting the conductive cable comprising actuating the conductive cable along a linear path within a pathway defined by the inner body.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof are placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a conductive cable;
(b) a pulley assembly, the pulley assembly comprising:
(i) an inner body defining a cable pathway extending between a proximal end and a distal end,
(ii) an outer body fixedly attached to the inner body, the outer body defining a cable port in communication with the proximal end of the cable pathway, and
(iii) a distal cap defining an interior; and
(c) an end effector electrically coupled to the conductive cable, comprising:
(i) a distal hook configured to receive therapeutic energy from the conductive cable and deliver the received therapy energy to tissue,
(ii) a proximal body fixed relative to the distal hook, wherein the proximal body defines a conductive wire channel in communication with the distal end of the cable pathway defined by the inner body,
wherein the conductive cable extends through the cable port of the outer body, the cable pathway of the inner body, and into the conductive wire channel,
wherein the proximal body defines a first port located within the interior of the distal cap, wherein the first port extends from an outer surface of the proximal body into the conductive wire channel.

2. The apparatus of claim 1, wherein the proximal body defines a second port located within the interior of the distal cap, wherein the second port extends from the outer surface of the proximal body into the conductive wire channel.

3. The apparatus of claim 2, wherein the second port is located proximally relative to the first port.

4. The apparatus of claim 1, further comprising a cap sealant located within the interior of the distal cap, wherein the cap sealant is interposed between the distal cap and the outer surface of the proximal body located within the interior.

5. The apparatus of claim 4, wherein the cap sealant mechanically isolates the distal cap from the end effector.

6. The apparatus of claim 1, further comprising a penetrating weld located within the first port, wherein the penetrating weld electrically couples the conductive cable with the end effector.

7. The apparatus of claim 1, further comprising a dielectric cable sealant located within the cable pathway of the inner body and the conductive wire channel.

8. The apparatus of claim 7, wherein the dielectric cable sealant is located within the first port.

9. The apparatus of claim 7, wherein the dielectric cable sealant extends from the proximal end to the distal end of the cable pathway.

10. The apparatus of claim 1, wherein the proximal body of the end effector further comprises a base defining a portion of the conductive wire channel, wherein the base comprises a semi-cylindrical shell having an interior surface and an exterior surface, wherein the inner body is directly attached to both the interior surface and the exterior surface of the base.

11. The apparatus of claim 10, wherein the base defines a window, wherein a portion of the cable pathway extends through the window.

12. The apparatus of claim 1, wherein the proximal body of the end effector comprises a scalloped surface, wherein the inner body of the pulley assembly comprises a complementary scalloped surfaced directly engaged with the scalloped surface of the end effector.

13. The apparatus of claim 1, wherein the inner body defines a though hole, wherein the outer body comprises a section extending within the through hole and directly engaging a portion of the inner body defining the through hole.

14. The apparatus of claim 1, wherein the inner body comprises a proximal dovetail, wherein the outer body comprises a section directly engaged with the dovetail of the inner body.

15. The apparatus of claim 1, wherein the inner body comprises a scalloped surface, wherein the outer body comprises a section directly engaged with the scalloped surface of the inner body.

16. An apparatus, comprising:
(a) a conductive cable;
(b) a pulley defining a cable pathway housing a portion of the conductive cable; and
(c) an end effector electrically coupled to the conductive cable, where in the end effector comprises:
(i) a distal hook portion extending distally from the pulley, wherein the distal hook portion is configured to receive therapeutic energy from the conductive cable and deliver the received therapy energy to tissue, wherein the distal hook portion comprises a first lateral thickness,
(ii) a proximal body attached to the pulley, wherein the proximal body comprises a second lateral thickness, wherein the second lateral thickness is greater than the first lateral thickness, and
(iii) a curved surface located between, and coupling together, the distal hook portion and the proximal body, wherein the curved surface is configured to define a controlled yield location.

17. The apparatus of claim 16, wherein the curved surface forms an annular surface.

18. The apparatus of claim 16, wherein the pulley comprises a conducive cable contact surface.

19. A method of manufacturing a monopolar hook and a pulley assembly, the method comprising:
(a) injection molding an inner body of the pulley assembly onto a proximal body of the monopolar hook;
(b) injection molding an outer body of the pulley assembly onto the inner body of the pulley assembly; and
(c) after injection molding the outer body of the pulley assembly, inserting a conductive cable within an interior of the monopolar hook and welding the conductive cable with the monopolar hook.

20. The method of claim 19, wherein inserting the conductive cable comprising actuating the conductive cable along a linear path within a pathway defined by the inner body.
